(19) United States
(12) Patent Application Publication
(10) Pub. No.: US 2022/0079984 A1
(43) Pub. Date: Mar. 17, 2022

(54) METHOD FOR TREATING CANCER WITH KIDNEY PROTECTION

(71) Applicant: Renibus Therapeutics, Inc., Southlake, TX (US)

(72) Inventors: Donald Jeffrey Keyser, Southlake, TX (US); Alvaro F. Guillem, Lantana, TX (US); Richard A. Zager, Seattle, WA (US)

(21) Appl. No.: 17/472,954

(22) Filed: Sep. 13, 2021

Related U.S. Application Data
(60) Provisional application No. 63/158,803, filed on Mar. 9, 2021.
(60) Provisional application No. 62/706,829, filed on Sep. 11, 2020.

(51) Int. Cl. A61K 33/26; A61K 39/395; A61K 31/7068; A61K 38/21; A61K 33/243; A61K 31/675; A61K 31/519; A61K 31/409; A61K 49/10; A61P 13/12

(52) U.S. Cl. CPC A61K 33/26; A61K 39/3955; A61K 31/7068; A61K 38/21; A61K 33/243; A61P 13/12; A61K 31/519; A61K 31/409; A61K 49/10; A61K 49/108; A61K 31/675

(57) ABSTRACT

The present invention involves a novel method for treatment of cancer that involves co-administration of iron sucrose and/or a protoporphyrin such as tin protoporphyrin in an amount sufficient to protect a patient's kidney against cytotoxicity of a chemotherapeutic agent.

### BACKGROUND

Cisplatin is a commonly used chemotherapy agent since the discovery of its anti-cancer activity more than 50 years ago. Currently, cis-diammine dichloroplatinum II (cisplatin) is being widely used clinically as an anticancer agent for testicular cancer, ovarian cancer, head and neck cancer, bladder cancer, and non-small-cell lung cancer. However, as it accumulates intensively in the kidneys and damages the kidneys, leading to serious toxic side effects, use of cisplatin has been limited.

Kidney protection has previously been confirmed through administration of FeS and SnPP. See U.S. Pat. No. 9,844,563 to Zager et al. However, the ability to effectively protect against renal damage during chemotherapy requires selective protection whereby a protective agent does not undermine the efficacy of the chemotherapy agent on tumor cells. Additional work is needed to develop effective protective agents for use during chemotherapy.

### DESCRIPTION OF THE DRAWINGS

FIG. 1 shows that RBT-3 (360 mg) induces rapid and marked increases in plasma hepcidin levels in human subjects.

FIG. 2 shows that RBT-3 (240 mg) induces rapid and marked increases in plasma hepcidin levels.

FIG. 3 shows that RBT-3 (120 mg) induces plasma hepcidin increases in both healthy volunteers (HV) and CKD subjects.

FIG. 4 shows that increases in plasma hepcidin levels over baseline values.

FIG. 5 shows that the Nrf2 pathway is activated by RBT-3 injection in CD-1 mice.

FIG. 6A-FIG. 6B show that RBT-3 induces prominent increases in heme oxygenase 1 (HO-1) expression in mouse proximal tubules, as assessed by immunohistochemistry.

### DETAILED DESCRIPTION

The present invention relates to the use of renal protective agents that selectively protect the kidney and/or liver relative to cancer cells, and methods of treating cancer using a chemotherapy agent, or screening for cancer using a radiocontrast agent using such renal protective agents. In one aspect, the renal protective agent is an iron composition such as iron sucrose (FeS). In another aspect, the renal protective agent is a protoporphyrin (e.g., tin protoporphyrin, SnPP). In another aspect, the invention involves administering a combination of iron composition and protoporphyrin during cancer chemotherapy or radiocontrast imaging. For example, administering iron sucrose and tin protoporphyrin along with a chemotherapy agent.

The present invention utilizes iron compositions such as iron sucrose such as RBT-3 is described in U.S. patent application Ser. No. 16/805,223, entitled “NOVEL IRON COMPOSITIONS AND METHODS OF MAKING AND USING THE SAME” filed Feb. 20, 2020, and claiming priority to U.S. provisional application 62/812,028 filed Feb. 20, 2019, the disclosure of which is incorporated herein in its entirety. The protoporphyrin includes metal porphyrins, such as tin protoporphyrin (SnPP). A number of synthetic analogs of iron protoporphyrin IX are known. These compounds are commercially available and/or can be readily synthesized by known methods. They include, for example, platinum, zinc, nickel, cobalt, copper, silver, manganese, chromium, and tin protoporphyrin IX. For convenience, these compounds can be referred to generically as Me-protoporphyrin or MePP, were Me stands for metal, and specifically by utilizing the chemical symbol for the metal such as Cr-protoporphyrin (CrPP), Sn-protoporphyrin (SnPP), Zn-protoporphyrin (ZnPP) for the chromium, tin, and zinc protoporphyrin compounds respectively.

Chemotherapy agents that can be used in connection with the present invention include compounds within the class of platinum analogs, including cisplatin, as well as carboplatin, eloxatin, or oxaliplatin.

Renal Protective Effects of Iron Compositions During Chemotherapy

Hepcidin is a well-recognized iron regulatory protein that is produced predominantly in hepatocytes in response to macrophage iron levels and pro-inflammatory states. Coffey R, Ganz T. Iron homeostasis: An anthropocentric perspective. J Biol Chem. 2017; 292:12727-12734. While it was initially considered to have antimicrobial properties Michels K, Nemeth E, Ganz T, Mehrad B. Hepcidin and host defense against infectious diseases, PLoS Pathog. 2015; 11(8):e1004998, in recent years it has been demonstrated to have acute kidney protective effects. Van Swelm R P, Wetzels J F, Verweij V G, et al. Renal handling of circulating and renal-synthesized hepcidin and Its protective effects against hemoglobin-mediated kidney Injury. J Am Soc Nephrol. 2016; 27:2720-2732; Swaminathan S. Iron homeostasis pathways as therapeutic targets in acute kidney injury. Nephron. 2018; 140:156-159; Scindia Y, Wlazlo E, Leeds J, et al. Protective role of hepcidin in polymicrobial sepsis and acute kidney injury. Front Pharmacol. 2019; 10:615. Published 2019 Jun. 6. doi:10.3389/fphar.2019.00615; Scindia Y, Dey P, Thirunagari A, Liping H, Rosin D L, Floris M, Mark D. Okusa M D, Swaminathan S. Hepcidin mitigates renal ischemia-reperfusion Injury by modulating systemic iron homeostasis. J Am Soc Nephrol. 2015; 26: 2800-2814; Wang X, Zheng X, Zhang J, Zhao S, Wang Z, Wang F, Shang W, Barasch J, Qiu A. Physiological functions of ferroportin in the regulation of renal iron recycling and ischemic acute kidney injury. Am J Physiol. 2018; 315: F1042-F1057. For example, recombinant hepcidin administration has been shown to mitigate experimental ischemic AKI.

Conversely, hepcidin deficient mice are highly susceptible to ischemic renal damage (6). The mechanism by which hepcidin exerts its protective actions remains speculative. However, most interest has focused on the following potential pathway (6,7): i) due to its small size (25 kDa), hepcidin undergoes rapid glomerular filtration, followed by proximal tubule endocytic uptake; ii) hepcidin binds to the iron exporter, ferroportin, causing its cellular redistribution and subsequent proteolytic destruction; iii) ferroportin loss may increase intracellular catalytic iron levels; and iv) a rise in cytosolic iron can stimulate the synthesis of ferritin which confers its well-known cytoprotective/antioxidant effects. Johnson A C, Gooley T, Guillem Keyser J A, Rasmussen H, Singh B, Zager R A. Parenteral iron sucrose-induced renal preconditioning: differential ferritin heavy and light chain expression in plasma, urine, and internal organs. Am J Physiol. 2019; 317: F1563-F1571; Zarjou A, Bolisetty S, Joseph R, et al. Proximal tubule H-ferritin mediates iron trafficking in acute kidney injury. J Clin Invest. 2013; 123:4423-4434. However, additional protective mechanisms may also be operative. As just one example, synergistic interactions between the hepcidin and the cytoprotective Nrf2 pathways may exist (10-13). Lim P J, Duarte T L, Arezes J, et al. Nrf2 controls iron homeostasis in haemochromatosis and thalassaemia via Bmp6 and hepcidin. Nat Metab. 2019; 1:519-531; Bayele H K, Balesaria S, Srai S K. Phytoestrogens modulate hepcidin expression by Nrf2: Implications for dietary control of iron absorption. Free Radic Biol Med. 2015; 89:1192-1202; Tanaka Y, Ikeda T, Yamamoto K, Ogawa H, Kamisako T. Dysregulated expression of fatty acid oxidation enzymes and iron-regulatory genes in livers of Nrf2-null mice. J Gastroenterol Hepatol. 2012; 27:1711-1717; Harada N, Kanayama M, Maruyama A, et al. Nrf2 regulates ferroportin 1-mediated iron efflux and counteracts lipopolysaccharide-induced ferroportin 1 mRNA suppression in macrophages. Arch Biochem Biophys. 2011; 508:101-109.

Given the burgeoning evidence that recombinant hepcidin can confer renal protection against AKI, we questioned whether a newly developed, novel IV iron sucrose formulation (RBT-3), with proven renal safety when administered to healthy volunteers and CKD patients can acutely stimulate hepcidin production and thus, increase renal hepcidin levels. RBT-3 is described in U.S. patent application Ser. No. 16/805,223, entitled “NOVEL IRON COMPOSITIONS AND METHODS OF MAKING AND USING THE SAME” filed Feb. 20, 2020, and claiming priority to U.S. provisional application 62/812,028 filed Feb. 20, 2019, the disclosure of which is incorporated herein in its entirety.

To explore these possibilities, we have administered different doses of RBT3 to healthy volunteers (HVs) and to subjects with stage 3-4 CKD, and measured plasma hepcidin concentrations over a 72 hr period. Urinary hepcidin levels were also assayed to confirm renal hepcidin delivery. In complimentary mouse experiments, the potential effects of RBT-3 on both hepatic and renal cortical hepcidin production were probed. Given the current interest in hepcidin-Nrf2 interactions, the impact of RBT-3 on Nrf2 activity was assessed. Finally, the ability of RBT-3 to confer protection against a clinically relevant AKI model, cisplatin nephrotoxicity, and potential mechanisms of action, were tested. The results of these complementary clinical and experimental studies form the basis of this report.

We have found that the cytotoxic effects of chemotherapy agents to the kidney may be reduced by co-administration of the chemotherapy with an iron composition. The co-administration of radiocontrast agents with an iron composition may also reduce cytotoxic effects of the radiocontrast agent. The iron composition is preferably an iron sucrose composition. The iron sucrose composition is preferably an iron sucrose composition comprising bicarbonate. In one example, the iron sucrose composition is RBT-3. The iron composition may have one or more of the following characteristics, including an Fe2+/Fe3+ ratio of approximately 1-10%, 2-5%, 3-4% or about 3.4%, a total iron content of 5-19 mg/ml, 8-18 mg/ml, 10-15 mg/ml, or about 12 mg/ml, and organic carbon content of 4-11%, 6-9% or about 7.7%, an osmolality of 1100-1600 mOsM/Kg, 1400-1580 mOsm/Kg, or about 1540 mOsm/Kg and iron core size of about 1-3 nm, 2-2.8 nm, or about 2.39 nm, a Na content of between 0.8%-3%, 1%-2%, or about 1.26%, and an average molecular weight of 10,000-30,000 daltons, 20,000-25,000 daltons, or about 23,881 daltons.

Hepicidin is a key regulator of systemic and intracellular iron homeostasis, and has recently been noted to possess renal tubular cytoprotective effects. As previously discussed, following recombinant hepcidin administration, renal filtration, proximal tubular uptake, and subsequent degradation of ferroportin result. Since ferroportin is the only known cellular exporter of iron, its degradation is believed to increase proximal tubular iron content which then stimulates ferritin production. Given that ferritin is a potent anti-oxidant, it may be a key arbiter of hepcidin's cytoprotective effects.

In two previously reported mouse studies, our laboratory demonstrated that within 18-24 hrs of Venofer FeS administration, renal protection against diverse forms of AKI (glycerol, maleate, or ischemia-reperfusion) results. Johnson A C M, Zager R A. Mechanisms and consequences of oxidant-induced renal preconditioning: an Nrf2-dependent, P21-independent, anti-senescence pathway. Nephrol Dial Transplant. 2018; 33:1927-1941; Johnson A C, Becker K, Zager R A. Parenteral iron formulations differentially affect MCP-1, HO-1, and NGAL gene expression and renal responses to injury. Am J Physiol 2010; 299: F426-F435.

We suggested that this was due, in part, to FeS-driven increases in proximal tubule ferritin content. Johnson A C, Gooley T, Guillem Keyser J A, Rasmussen H, Singh B, Zager R A. Parenteral iron sucrose-induced renal preconditioning: differential ferritin heavy and light chain expression in plasma, urine, and internal organs. Am J Physiol. 2019; 317: F1563-F1571. However, Venofer FeS gains minimal proximal tubule luminal access. Zager R A, Johnson A C, Hanson S Y. Parenteral iron nephrotoxicity: potential mechanisms and consequences. Kidney Int. 2004; 66: 144-156. This suggests an alternative possibility for FeS driven ferritin increases: that FeS might stimulate hepatic hepcidin synthesis which then evokes renal ferritin production by the above noted pathway. However, it remained unclear as to whether FeS can evoke rapid and sustained hepcidin production (i.e., within a 24 hr period), such as would be required for it to contribute to our previously noted FeS-induced preconditioning state.

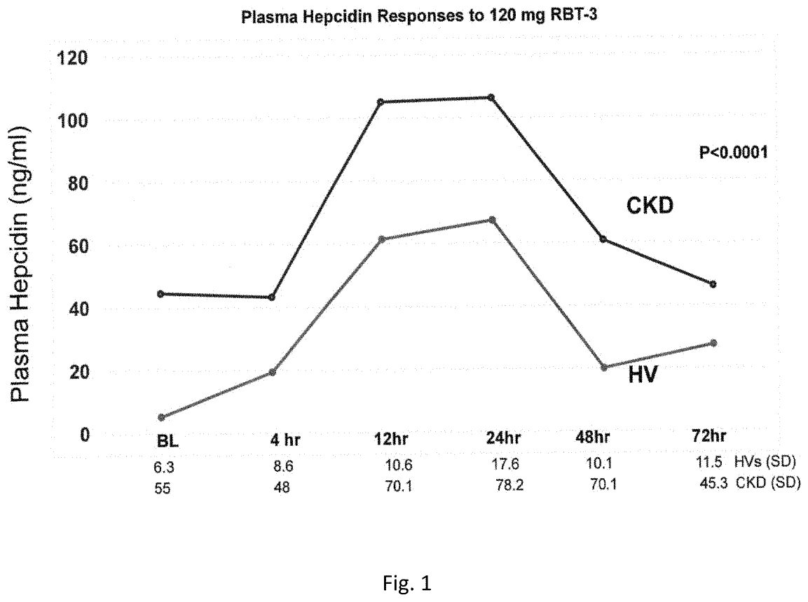

To address this issue, we have assessed the impact of a novel FeS, “RBT-3” on hepcidin production in healthy human subjects and patients with advanced CKD. We selected this iron formulation for study based on preliminary evidence from our laboratory that RBT-3 induces a more robust cytoprotective action than does equimolar Venofer. To gain additional insights, RBT-3 effects on hepicidin expression in normal mice was assessed. As shown in FIGS. 1-3, in both healthy humans and CKD subjects, rapid, dose dependent, plasma hepcidin increases were observed following RBT-3 injection. This response peaked at 24 hrs, with both subject groups manifesting ^{˜}15 fold plasma hepcidin increases in response to 240 or 360 mg RBT-3 injection. At baseline, plasma hepcidin levels were elevated in CKD subjects, compared to healthy volunteers, likely reflecting CKD's pro-inflammatory state. Honda H, Hosaka N, Ganz T, Shibata T. Iron metabolism in chronic kidney disease patients. Contrib Nephrol. 2019; 198:103-111; Weiss G, Ganz T, Goodnough L T. Anemia of inflammation. Blood. 2019; 133: 40-50.

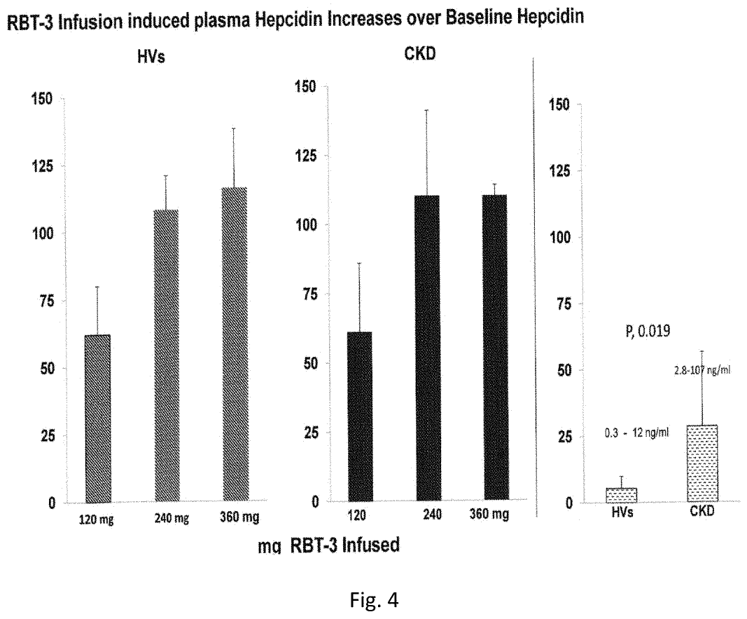

This raised the theoretical possibility that in CKD patients, hepcidin increases were already maximal, precluding further RBT-3 mediated hepcidin increases. However, this was clearly not the case given that absolute hepcidin elevations over baseline values were virtually identical in the HV and CKD study groups (FIG. 4). Of interest, the 24 hr peak values fell by ^{˜}50% within 48-72 hr post RBT-3 injection. This presumably reflected declining hepcidin production in concert with rapid renal hepcidin uptake and urinary hepcidin excretion. Indeed, the latter was evidenced by′ 4 fold increases in urinary hepicidin concentrations in both the CKD and healthy volunteer groups at 24 hrs post RBT-3 injection.

Hepatic hepicidin production in response to iron loading is thought to exclusively result from increased gene transcription, initiated by holotransferrin binding to its hepatic receptors (Tfr1, Trf2). Subsequent BMP-SMAD pathway activation then up-regulates HAM P1 gene transcription. To confirm that this pathway was activated by RBT-3 injection, we measured HAMP1 mRNA in both mouse liver and kidney at 24 hrs post RBT-3 administration. Surprisingly, whereas marked HAM P1 mRNA increases were observed in both organs, a 10 fold greater response was observed in kidney. To our knowledge, preferential renal vs hepatic HAMP1 induction in response to Fe has not previously been reported. This raises the intriguing possibility that FeS/RBT-3 may trigger renal hepcidin loading by both indirect (hepatic production) and direct (renal derived) mechanisms.

To expand our understanding of the scope of FeS/RBT-3's protective actions, we have now tested whether it can be expressed against experimental cisplatin-induced ARF. This model was chosen for study in light of the following four considerations: First, unlike our previously tested AKI models (ischemia, maleate, glycerol-induced rhabdomyolysis) which are fully apparent within ^{˜}24 hrs, cisplatin nephrotoxicity evolves slowly, requiring at least 3 days for the full expression of renal failure. Thus, it was unclear as to whether an FeS/RBT-3 mediated protective effect could be expressed over this prolonged time frame. Second, cisplatin adducts induce early and prominent DNA damage (e.g., DNA cross linking), culminating in apoptotic or necrotic cell death. Miller R P, Tadagavadi R K, Ramesh G, Reeves W B. Mechanisms of cisplatin toxicity. Toxins. 2010; 11: 2490-2518.

Given this unique injury-initiating event, it was unclear whether RBT-3 could still confer a protective effect. Third, cisplatin remains a widely used chemotherapeutic agent, with a 25-30% clinical AKI rate. Latcha S, Jaimes E A, Patil S, Glezerman I G, Mehta S, Flombaum C D. Long-term renal outcomes after cisplatin treatment. Clin J Am Soc Nephrol. 2016; 11:1173-1179. Thus, identifying a protective mechanism to mitigate cisplatin toxicity could fill a currently unmet medical need; and fourth, cisplatin administration is a scheduled clinical event. Hence, RBT-3 could be administered ^{˜}18-24 hrs prior to cisplatin infusion, thereby allowing the necessary time for the full development of an FeS-mediated cytoresistant state. Indeed, when RBT-3 was administered to mice 18 hrs prior to cisplatin injection, marked renal protection was observed, as evidenced by steep reductions in BUN, plasma creatinine, and plasma NAG concentrations (Table 3). Thus, these findings add further support for the concept that FeS/RBT-3 preconditioning can exert broad based, and potentially clinically applicable, renal protective effects.

A fourth, and completely unexpected, protective action evoked by RBT-3 preconditioning was a dramatic suppression in renal cisplatin uptake. Because cisplatin nephrotoxicity is highly dependent on proximal tubule cell uptake, the observed 40% reduction in renal cortical cisplatin levels must have played a dominant role in RBT-3's protective action. Proximal tubule cell cisplatin uptake is mediated via organic cation transporters (e.g. OCT2, MATE1), located in the basolateral membrane. While OCT2 transport is the prime determinant of proximal tubule cisplatin uptake, it has recently been suggested that MATE1 (“multidrug and toxin extrusion protein 1”) can also evoke luminal cisplatin efflux (24). How RBT-3 impacts these cellular uptake and efflux pathways remains unknown. However, one might speculate that a hepcidin-MATE-1 interaction might increase proximal tubule cisplatin efflux. Spreckelmeyer S, van der Zee M, Bertrand B, Bodio E, Sturup S, Casini A. Relevance of copper and organic cation transporters in the activity and transport mechanisms of an anticancer cyclometallated gold(III) compound in comparison to cisplatin. Front Chem. 2018; 6:377; Nieskens T T G, Peters J G P, Dabaghie D, et al. Expression of organic anion transporter 1 or 3 in human kidney proximal tubule cells reduces cisplatin sensitivity. Drug Metab Dispos. 2018; 46: 592-599.

In a number of previous publications, our laboratory has demonstrated clear differences in kidney biologic responses to different parenteral iron formulations, including iron dextran, iron gluconate, venofer, and ferumoxytol. These differences include varying degrees of toxicity, severities of oxidative stress, and potential pro-inflammatory effects. Most notably, only FeS (both Venofer, and now RBT-3) have been shown to induce a renal preconditioning effect. Indeed, in unpublished work, we have found that although both Venofer and RBT3 each mitigate the glycerol AKI model, RBT-3 confers an approximately 35% greater renal protective effect. We now demonstrate that RBT-3 induces 5-fold greater renal HAMP1 response to RBT3 vs. Venofer. Furthermore, RBT3 induced twice the renal HO-1 mRNA response vs Venofer. Thus, Venofer and RBT3 clearly exert quantitatively different renal cytoprotective protein responses. What physicochemical difference(s) between Venofer vs. RBT-3 account for these differences remain unknown. However, these data clearly indicate that not all Fe preparations are alike and as of this time, RBT3 appears to offer the greatest cytoprotective influence.

We have demonstrated that renal hepcidin loading can be markedly (15×), and rapidly (<24 hrs) induced by IV RBT-3 injection. However, it should be noted that in addition to its impact on hepcidin, RBT-3 exert additional protective actions that cannot be reproduced by recombinant hepcidin injection. Two of the most promising are: 1) Nrf2 pathway activation within proximal tubules, documented for the first time in the present study and 2) RBT-3′ ability to blunt tubule nephrotoxin uptake (or accumulation), at least in the case of cisplatin. Given these considerations, it seems clear that RBT-3-induced preconditioning is mediated via multiple cellular pathways above and beyond hepcidin loading. That FeS has been shown to have an excellent clinical safety profile in humans further suggests potential clinical value as a renal protective/preconditioning agent. Finally, the data confirm that not all Fe sucrose preparations exert the same biologic effects. With the data gathered to date, RBT3 appears to be the most promising for inducing a renal preconditioning state.

Clinical Study

Nine healthy volunteers (HVs; eGFRs>70 ml/min/1.73 m2) and 9 patients with stage 3-4 CKD (15-59 ml/min/1.73 m2) were recruited for this investigation. The subjects that formed the basis for the current study were also the foundation of a previous study which evaluated differential effects of FeS on heavy and light chain ferritin expression (8). The study received IRB approval from Advarra IRB, Columbia, Md., and informed consent was obtained from each subject. The study conformed to the Helsinki Declaration. Exclusion criteria included pregnancy, any significant medical illness other than the presence of CKD, iron administration in the prior 30 days, or a plasma ferritin concentration of >500 ng/ml. Specific demographic data, screening eGFRs (CKD-EPI equation), BUNs, serum creatinines, and blood pressures for the two study groups are summarized in Table 1. More detailed information has previously be presented. Johnson A C, Gooley T, Guillem Keyser J A, Rasmussen H, Singh B, Zager R A. Parenteral iron sucrose-induced renal preconditioning: differential ferritin heavy and light chain expression in plasma, urine, and internal organs. Am J Physiol. 2019; 317: F1563-F1571. The trial was entered into Clinicialtrials.gov (NCT04072432).

For the purpose of this study, a novel FeS formulation, “RBT-3”, manufactured by Cascade Custom Chemistry; Portland, Oreg., was employed. A number of physicochemical differences between RBT-3 and the widely used FeS preparation “Venofer” exist (unpublished data). These include the following (RBT3 vs. Venofer, respectively): lower Fe2+/Fe3+ ratios; 3.4% vs 15.8%; lower total Fe content, 12 mg/ml vs. 20 mg/ml; lower organic carbon content; 7.7% vs. 12.14%; lower osmolality 1540 vs 1681 mOsm/Kg; smaller iron core size 2.39 vs 3.88 nm; higher Na content 1.26% vs. 0.5%; and lower average molecular weight, 23,881 vs. 31,335 daltons. In addition to these structural differences, quantitative differences in select biologic responses to RBT-3 and Venofer have also been noted (see Discussion).

The HV and CKD groups were each divided into 3 equal cohorts (n, 3 each), with each cohort receiving either 120, 240, or 360 mg of RBT-3 (12 mg/ml stock solution). The RBT-3 dose (10, 20, or 30 ml of stock solution) was infused IV with 100 ml of saline over 2 hr. The subjects remained overnight at the study site (Riverside Clinical Research, Edgewater, Fla.) to screen for potential adverse events. Timed heparinized plasma samples were collected at baseline (0) and at 4, 12, 24, and 72 hrs. ‘Spot’ urine samples were obtained at baseline and 24 hrs following RBT-3 infusion. The samples were assayed in duplicate for hepcidin using a commercially available ELISA (R & D Systems; Minneapolis, Minn.; kit #DY8307). Urine hepcidin values were factored by urine creatinine concentrations. ELISA standard curve samples were provided by the manufacturer.

Mouse Experiments

1. RBT-3-induced hepcidin expression in mice. Male CD-1 mice (35-40 gms; Charles River Labs, Wilmington, Mass.) were used for all animal studies which were approved by the institution's IACUC. Mice were injected via the tail vein with either 1 mg of RBT-3 or vehicle (n, 5 each). Eighteen hrs later, they were deeply anesthetized with pentobarbital (40-50 mg/Kg), the abdominal cavities were opened, a blood sample was obtained from the vena cava, and then the kidneys and livers were quickly resected, iced, and total RNA and protein were extracted (14). Renal cortical and hepatic hepcidin (HAMP1) mRNA levels were measured by competitive RT-PCR using the following primer pair: left: cagcagaacagaaggcatga; right: agatgcagatggggaagttg. The mRNA values were factored by simultaneously determined GAPDH product (14). Plasma, hepatic, and renal cortical hepcidin levels were also determined by ELISA (14).

2. RBT-3 effects on mouse kidney Nrf2 expression. To assess whether RBT-3 activates the Nrf2 pathway, in addition to its effects on hepcidin, 5 control and 5 RBT-3 treated mice had kidney samples collected 4 hrs following injections. Total mRNA was extracted and assayed for 4 Nrf2 activated genes: 1) heme oxygenase 1 (HO-1); 2) NAD(P)H quinone dehydrogenase 1 (NQO1); 3) sulfiredoxin-1 (SRXN1); and 4) glutamate-cysteine-ligase-catalytic subunit (GCLC) by RT-PCR, as previously described (15). In addition, Nrf2 nuclear translocation was assessed by extracting nuclear protein and assaying for Nrf2 by ELISA, as previously performed by this laboratory (15).

3. Immunohistochemistry analysis of RBT-3 effects on proximal tubule HO-1 expression. To confirm that RBT3 mediated Nrf2 activation resulted in increased Nrf2 sensitive protein levels in proximal tubule cells, HO-1 protein expression was assessed in kidneys obtained 18 hrs post RBT-3 or vehicle injection. Formalin-fixed paraffin-embedded tissues were sectioned at 4 microns onto positively-charged slides and baked for 1 hour at 60° C. The slides were then dewaxed and stained on a Leica BOND Rx stainer (Leica, Buffalo Grove, Ill.) using Leica Bond reagents for dewaxing (Dewax Solution), antigen retrieval (Epitope Retrieval Solution 2), and rinsing after each step (Bond Wash Solution). Antigen retrieval was performed for 20 minutes at 100° C. with all other steps at ambient temperature. Endogenous peroxidase was blocked with 3% H₂O₂ for 5 minutes followed by protein blocking with TCT buffer (0.05M Tris, 0.15M NaCl, 0.25% Casein, 0.1% Tween 20, 0.05% ProClin300 pH 7.6) for 10 minutes. The primary antibody, HO-1 (Abcam ab189491) was applied for 60 minutes followed by the Leica anti-rabbit HRP polymer for 10 minutes and the application of the tertiary TSA-amplification reagent (PerkinElmer OPAL 650 at 1:100) for 10 minutes. Slides were removed from the stainer and stained with DAPI for 5 minutes, rinsed, and cover slipped with Prolong Gold Antifade reagent (Invitrogen/Life Technologies, Grand Island, N.Y.).

4. Impact of RBT-3 preconditioning on cisplatin nephrotoxicity and renal cisplatin uptake. Mice were injected with 1 mg RBT-3 or saline vehicle via the tail vein (n, 5 each). Eighteen hrs later, all mice were injected with cisplatin (15 mg/Kg; IP). Free food and water were provided throughout. Three days post injections, the mice were deeply anesthetized with pentobarbital (50 mg/Kg IP), blood samples were obtained from the inferior vena cava for BUN and creatinine concentrations. Plasma levels of the proximal tubule enzyme, N-acetyl glucosamidase (“NAG”), were also assessed (BioAssay Systems #DNAG-100; Hayward, Calif.). In addition, kidney cortical extracts were prepared in distilled water and cisplatin concentrations were determined (ProFoldin; Goettingen, Germany) according to the manufacturer's instructions.

5. Comparison between RBT3 and Venofer. To demonstrate that, in addition to structural differences, functional differences between RBT3 and Venofer, we compared hepcidin mRNA and HO-1 mRNA responses between the two iron sucrose preparations. Six mice were injected with 1 mg of either RBT3 or Venofer and 24 hrs later, liver and kidney mRNA samples were obtained. Six normal samples provided control values.

Statistics. Each sample was assayed in duplicate with the average being used for data presentation and analysis. The human hepcidin data are presented as means±1 SD. Baseline plasma hepcidin values for the HV and CKD groups were compared by unpaired Student's t test. Comparisons of hepcidin levels over time were analyzed by ANOVA for repeated measures with 95% confidence intervals being presented. Baseline demographic data were compared by unpaired Student's t test. Mouse data are given as means±1 SEM. Significance was judged by a p value of <0.05.

Baseline subject information. Selected demographics for the study population have previously been presented in detail. Table 1 provides a synopsis.

TABLE 1
Overview of healthy subjects and subjects with Stage 3-4 CKD (from Ref. 8).

| | | | | BP | BP | | | |
|---|---|---|---|---|---|---|---|---|
| Group | Gender | Age | Weight | (Syst) | (Diast) | eGFR | BUN | PCr |
| HV (9) | 66% | 52 ± 6 | 92 ± 10 | 125 ± 6 | 80 ± 2 | >70 | 14 | 1.0 ± 0.2 |
| | Male | | | | | | | |
| CKD (9) | 22% | 69 ± 4 | 94 ± 9 | 130 ± 2 | 75 ± 7 | 38 ± 8 | 25 | 1.5 ± 0.4 |
| | Male | | | | | | | |
| P value | | <0.001 | 0.71 | 0.43 | 0.18 | <0.001 | 0.013 | 0.005 |

Table 1 legend. Baseline characteristics of the 9 healthy volunteers (HVs) and 9 subjects with stage 3-4 CKD that formed the basis of the current study. The means and 95% confidence intervals range = mean ± the numbers in the parentheses. P values were derived by unpaired Student’s t test.

The CKD subjects were significantly older than the HVs, and there were fewer CKD males compared to the HV group. Mean eGFR for the CKD group was 38±8 ml/min/1.73 m² (^{˜}half that of the HV group which was >70 ml/min·1.73 m²). The lower eGFRs for the CKD subjects were manifested by elevated baseline and plasma creatinine concentrations, vs. those seen in the HVs.

Baseline and post RBT-3 plasma hepcidin concentrations in HV and CKD subjects. Consistent with the literature (16,17), CKD subjects had elevated baseline plasma hepcidin levels, vs. HVs. (26±25 ng/ml vs. 4±5 ng/ml, respectively; p=0.001). This presumably reflected a CKD associated pro-inflammatory state, and possibly decreased renal hepcidin clearance due to CKD-associated GFR reductions.

All subjects manifested dramatic, and dose dependent, plasma hepcidin increases in response to RBT-3 injection (FIGS. 1-3). The increases were observed as early as 4 hrs after RBT-3 injection, and reached peak values by 24 hrs (FIGS. 1-3). With the 240 and 360 mg dosages, the 24 hr plasma hepcidin levels were approximately 15× higher than their baseline values. After 24 hrs, rapid plasma hepcidin declines were observed. However, at the final assessed time point (72 hrs), ^{˜}2-4 fold plasma hepcidin increases over baseline values were still observed (all groups, p<0.001).

Despite the fact that CKD was associated with increased baseline hepcidin levels, these increases did not impact the degree to which RBT-3 caused plasma hepcidin increases. For example, the 24 hr increases over basal values were nearly identical for the HV and CKD groups whether the subjects received the 120, 240 or 360 mg RBT-3 dosage (FIG. 4).

Results

Human urinary hepcidin concentrations in response to RBT-3 injection. At baseline, urinary hepcidin concentrations did not significantly differ between the HV and CKD groups (47±36 vs 51±72 ng/mg urine creatinine, respectively). By 24 hrs post RBT-3 injection, both groups showed ^{˜}4× urinary hepicidin increases over baseline values (HVs, 219±104; p=0.04 vs baseline; CKD 176±93; p=0.014 vs baseline). Hence, these urinary increases are consistent with increased filtration of circulating hepcidin levels or possibly hepcidin efflux from tubules.

Mouse Experiments.

1. Mouse HAM P1 gene expression; plasma, renal, and hepatic hepcidin responses to RBT-3.

Baseline mouse hepatic HAMP1 mRNA expression (Table 2) was approximately 25× greater than that observed in renal cortex, consistent with the liver being the dominant site of hepcidin production (1). While both liver and kidney each responded to RBT-3 injection with HAM P1 mRNA increases, the degree of the HAMP1 mRNA increase was dramatically greater in kidney vs. the liver (by a factor of 10×). However, baseline and post RBT-3 hepcidin protein levels were comparable for the two organs (^{˜}5× elevations over baseline values; see Table 2). These increases corresponded with an approximate 3 fold RBT-3 induced increase in plasma hepcidin levels (Table 2).

TABLE 2
Hepcidin expression in mouse liver, kidney, and plasma under control
conditions and 18 hrs following RBT-3 administration.

| | | | | Hepatic | Kidney |
|---|---|---|---|---|---|
| | Hepatic | Kidney | Plasma | Hepcidin | Hepcidin |
| | HAMP1 | HAMP1 | Hepcidin | (ng/mg | (ng/mg |
| Group | mRNA | mRNA | (ng/ml) | protein) | protein) |
| Control | 27 ± 4 | 0.9 ± 0.2 | 359 ± 53 | 2.0 ± 0.3 | 1.4 ± 0.1 |
| 24 hr post | 46 ± 6 | 578 ± 198 | 1057 ± 178 | 6.2 ± 0.4 | 6.6 ± 1.5 |
| RBT-3 | | | | | |
| P value | =0.028 | =0.01 | <0.001 | <0.001 | <0.005 |

Table 2 legend.
The two groups were comprised of 5 mice per group.
The HAMP1 mRNAs are factored by GAPDH.
The values are means ± 1 SEM.
P values were derived by unpaired Student's test.

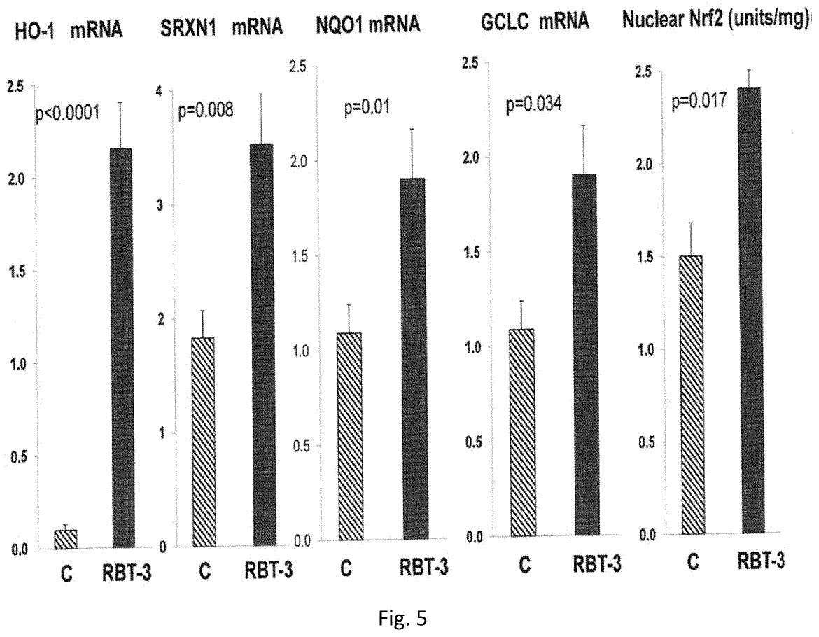

2. Mouse renal cortical Nrf2 gene activation in response to RBT-3. Each of the 4 tested Nrf2 responsive genes manifested significant increases in their mRNAs following RBT-3 injection (see FIG. 5). The most responsive gene appeared to be HO-1, given ^{˜}20 fold HO-1 mRNA increases vs. control levels. In addition, a significant increase in nuclear Nrf2 protein levels were observed in the RBT-3 treated group, compared to controls (p=0.017).

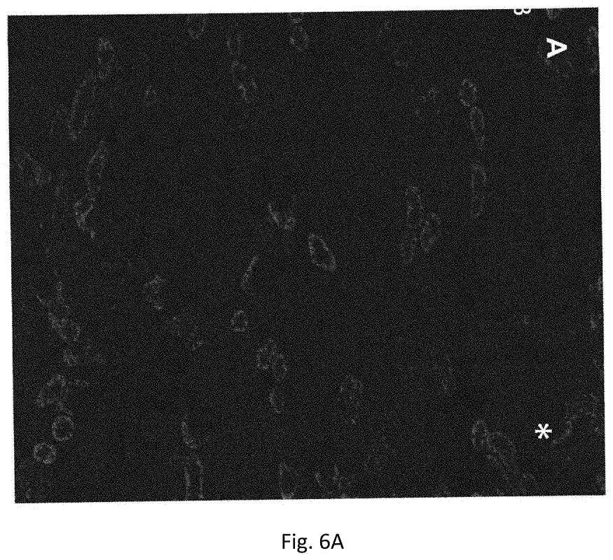

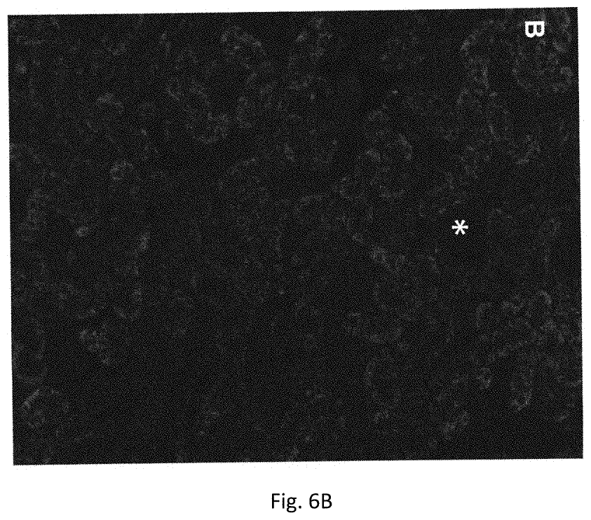

3. HO-1 immunofluorescence. Focal proximal tubule HO-1 staining was observed in control kidney tissue (FIG. 6). The extent and intensity of HO-1 staining was greatly increased 18 hrs following RBT-3 injection. The increase appeared to be largely confined to proximal tubule cells, which manifested intense cytoplasmic staining. No apparent increase in glomerular or medullary HO-1 staining was observed in response to RBT-3 injection.

4. RBT-3 effects on cisplatin nephrotoxicity. By 3 days post cisplatin injection, severe renal injury was apparent in control mice, as denoted by marked BUN and plasma creatinine elevations (Table 3). RBT-3 preconditioning caused an approximate 50% reduction in cisplatin-induced injury, as assessed by BUN and creatinine levels. That this protection reflected decreased proximal tubule injury was denoted by an almost complete blockade of NAG release into the systemic circulation (Table 3). Of interest, the RBT-3 mediated protection was associated with (and likely due, at least in part) to a 40% reduction in renal cortical cisplatin concentrations (Table 3).

TABLE 3
Cisplatin induced AKI: Impact of RBT-3 preconditioning.

| | BUN | Creat | Plasma | Tissue |
|---|---|---|---|---|
| Group | (mg/dl) | (mg/dl) | NAG | Cisplatin |
| Cisplatin | 105 ± 11 | 0.7 ± 0.1 | 19.5 ± 0.8 | 227 ± 35 |
| Cisplatin/ | 48 ± 5 | 0.43 ± 0.03 | 9.8 ± 1.2 | 142 ± 9 |
| RBT-3 | | | | |
| P value | 0.001 | 0.001 | =0.0025 | 0.048 |
| Normal | 24 ± 1 | 0.3 ± 0.03 | 8.9 ± 2.0 | 0 |
| values | | | | |

Table 3 legend.
The values are presented as means ± 1 SEM.
BUN and creatinine values are mg/dL.
Plasma NAG values are activity units/liter.
Cisplatin values are μg/gram tissue wet weight.
Statistical comparisons were made by unpaired Student's t test.

5. Comparison between Venofer and RBT3 FeS preparations. Both liver and kidney showed significant increases in HAMP1 mRNA values following injection of RBT-3. However, the increase in renal HAM P1 mRNA was far greater with RBT3 (11× vs 2× increases with RBT-3 vs. Venofer (p=0.015; Table 4). In contrast, whereas both irons raised hepcidin mRNAs, there was no difference in hepcidin protein concentration observed in liver with Venofer vs RBT-3. This indicates a renal specific HAMP1 augmented response. HO-1 mRNA was also probed in kidney samples and the increase was twice as high with the RBT3 vs Venofer treatment. These data clearly demonstrated biologic response differences between the two FeS preparations.

TABLE 4
Comparisons between Venofer FeS vs. RBT-3 FeS

| | Renal | | Renal |
|---|---|---|---|
| | HAMP1 | Hepatic | HO-1 |
| Group | mRNA | mRNA | mRNA |
| Venofer | 1.48 ± 0.5 | 4.4 ± 0.3 | 2.2 ± 0.3 |
| | (2.2×) | | |
| RBT-3 | 7.31 ± 2.28 | 4.8 ± 0.4 | 4.4 ± 0.4 |
| | (11×) | | |
| P value | =0.015* | NS | <0.025 |
| | | (0.48) | |
| Normal | 0.67 ± 0.02 | 2.9 ± 0.5 | 0.8 ± 0.1 |
| values | | | |

Table 4 legend.
Mice were injected with 1 mf Venofer FeS or RBT-3 FeS, followed by assessments of HAMP1 mRNA and HO-1 mRNA 24 later.
RBT-3 induced an 11 fold increase in HAMP1 (hepcidin) mRNA vs a 2.2 fold increase with Venofer.
RBT-3 also induced a doubling of HO-1 mRNA, compared to Venofer.
P = 0.015 after log base 10 data conversion.
Non converted, p = 0.037.

FIG. 1. RBT-3 (360 mg) induces rapid and marked increases in plasma hepcidin levels in human subjects. Within 4 hrs of RBT-3 injection (360 mg), plasma hepicidin increases were observed and peaked at 24 hrs (^{˜}15× baseline). Nearly identical responses were observed in the healthy volunteer (HV) and CKD cohorts. The 95% confidence intervals (±) for the HV and CKD groups are given in the table at the bottom of the figure. P value derived from ANOVA for repeated measures. HV=healthy volunteers; CKD=chronic kidney disease.

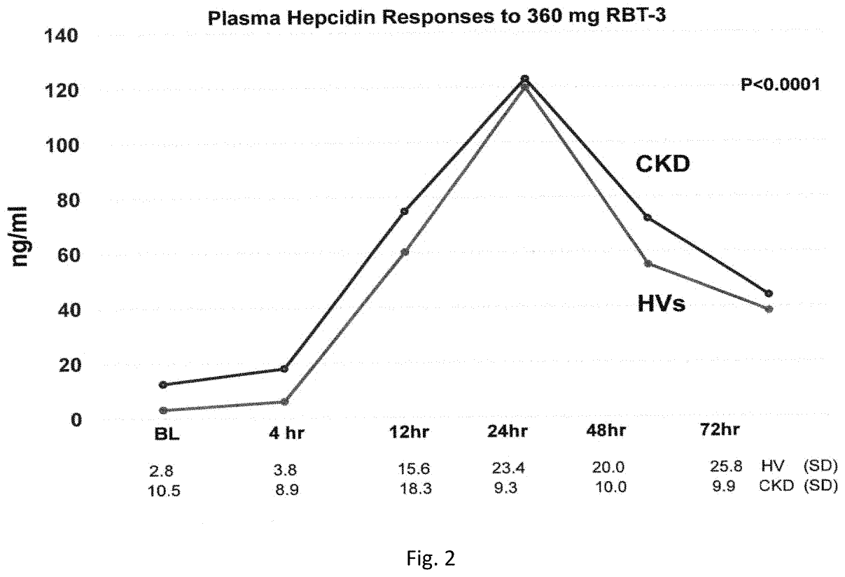

FIG. 2. RBT-3 (240 mg) induces rapid and marked increases in plasma hepcidin levels. As was the case with the 360 mg dose, hepcidin increases were observed at 4 hrs and peaked at the 24 hr time point. No significant differences were observed between the CKD and healthy volunteer (HV) groups. The 95% confidence intervals (±) for the healthy volunteers (HV) and CKD groups are given in the table at the bottom of the figure. P value derived from ANOVA for repeated measures.

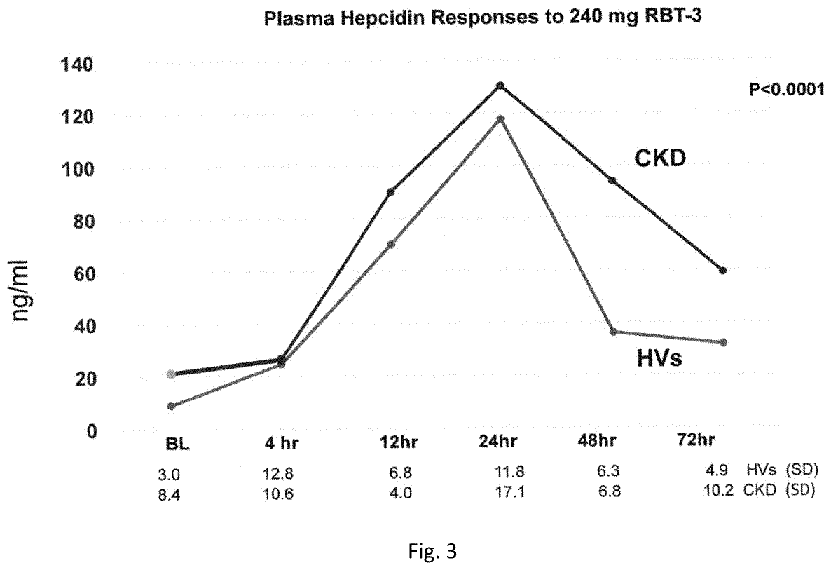

FIG. 3. RBT-3 (120 mg) induces plasma hepcidin increases in both healthy volunteers (HV) and CKD subjects. The CKD subjects had higher plasma hepcidin levels throughout the 72 hr period, compared to the HVs. This presumably reflects the higher baseline (starting) hepcidin levels in the CKD group (see text). This was true throughout the 360 mg, 240 mg and the 120 mg RBT-3 treatment groups (see text and right hand panel of FIG. 4).

FIG. 4. Increases in plasma hepcidin levels over baseline values. Because baseline plasma hepcidin levels were elevated in the CKD cohort, compared to healthy volunteers (HVs; see right hand panel), the RBT-3-induced increases over these baseline values were calculated (24 hr peak values−baseline values). This allowed comparison between the CKD and healthy volunteer hepcidin responses. As is apparent, the HVs and CKD groups manifested highly comparable hepcidin increases with each iron dose. Values are means±95% confidence intervals. The 240 mg and 360 mg RBT-3 dose induced comparable hepcidin increases, both of which were greater than those observed in the 120 mg RBT-3 dose groups.

FIG. 5. The Nrf2 pathway is activated by RBT-3 injection in CD-1 mice. At 4 hrs following RBT-3 injection into mice, significant increases in each of the 4 tested Nrf2 responsive gene mRNAs were observed (compared to simultaneously studied control mice). Further evidence of Nrf2 pathway activation was the finding of increased nuclear Nrf2 protein binding in nuclear protein extracts. Heme oxygenase 1 (HO-1); NAD(P)H quinone dehydrogenase 1 (N0,01); sulfiredoxin-1 (SRXN1); and glutamate-cysteine-ligase-catalytic subunit (GCLC).

FIG. 6. RBT-3 induces prominent increases in heme oxygenase 1 (HO-1) expression in mouse proximal tubules, as assessed by immunohistochemistry. The control kidney, shown in the left hand panel, demonstrates variable cytoplasmic staining of proximal tubule segments. In contrast, the kidney harvested 18 hrs post FeS injection (right hand panel) showed a prominent, near confluent proximal tubule HO-1 increase. In contrast, no increased HO-1 staining was apparent in glomeruli, depicted by the asterisks. Thus, these findings confirmed that the HO-1 mRNA changes depicted in FIG. 5, were reflected by: i) increased HO-1 protein levels, and ii) that these increases occurred within proximal tubules.

Renal Protective Effects of Protoporphyrins During Chemotherapy

SnPP (1 umole) or vehicle (V) was given on day zero, 15 mg/kg of cisplatin was given on day 1, and BUN, creatinine, renal cortical LDH, and the mRNAs for NGAL, MCP-1, IL-6, HO-1, and p21 were measured 3 days post cisplatin injection. SnPP conferred statistically significant protection in all cisplatin treated mice, compared to cisplatin alone P<0.01).

| | | P | Tissue | NGAL | MCP-1 | IL-6 | HO-1 | P21 |
|---|---|---|---|---|---|---|---|---|
| Group | BUN | creatinine | LDH | mRNA | mRNA | mRNA | mRNA | mRNAs |
| Controls | 26 ± 1 | 0.28 ± 0.02 | 1000 ± 29 | 0.03 ± 0.0 | 0.31 ± 0.05 | 0.01 ± 0.0 | .30 ± 0.07 | 0.34 ± 0.07 |
| Cisplatin (V) | 103 ± 22 | 0.94 ± 0.41 | 754 ± 54 | 16.9 ± 12 | 10.9 ± 5.7 | 2.51 ± 1.65 | 1.16 ± 0.28 | 4.23 ± 0.61 |
| SnPP/Cislatin | 30 ± 3 | 0.32 ± 0.01 | 890 ± 20 | 0.88 ± 0.25 | 1.38 ± 0.16 | 0.07 ± 0.02 | 0.56 ± 0.03 | 2.1 ± 0.17 |

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

1. A method for protecting the kidney during cancer chemotherapy comprising:
administering a chemotherapeutic agent and an amount of iron composition to a human patient, wherein the amount of iron composition confers protection of the patent's kidney against cytotoxic effects of the chemotherapeutic agent.

2. The method of claim 1, wherein the chemotherapeutic agent is bevacizumab, gemcitabine, IFN therapy, cisplatin, ifosfamide, pemetrexed, cetuximab, or methotrexate, or a combination thereof.

3. The method of any of claim 1, wherein the chemotherapeutic agent is cisplatin.

4. The method of claim 1, wherein the method induces an increase in hepcidin in the human patient.

5. The method of claim 1, wherein the iron composition is an iron sucrose composition.

6. The method of claim 5, wherein the iron sucrose composition comprises bicarbonate.

7. The method of claim 5, wherein the iron sucrose composition comprises Fe2+/Fe3+ ratio of approximately 1-10%, a total iron content of 5-19 mg/ml, an organic carbon content of 4-11%, an osmolality of 1100-1600 mOsM/Kg, an iron core size of about 1-3 nm, a Na content of between 0.8%-3%, and an average molecular weight of 10,000-30,000 daltons.

8. The method of claim 5, wherein the iron sucrose composition comprises Fe2+/Fe3+ ratio of approximately 3-4%, a total iron content of 10-15 mg/ml, an organic carbon content of 6-9%, an osmolality of 1400-1580 mOsm/Kg, an iron core size of about 2-2.8 nm, a Na content of between 1%-2%, and an average molecular weight of 20,000-25,000 daltons.

9. The method of claim 5, wherein the iron sucrose composition comprises Fe2+/Fe3+ ratio of approximately about 3.4%, a total iron content of about 12 mg/ml, an organic carbon content of about 7.7%, an osmolality of about 1540 mOsm/Kg, an iron core size of about 2.39 nm, a Na content of about 1.26%, and an average molecular weight of about 23,881 daltons.

10. A method for protecting the kidney during radiocontrast imaging comprising:
administering the radiocontrast agent and an amount of iron composition to a human patient, wherein the amount of iron sucrose confers protection of the patent's kidney against cytotoxic effects of the radiocontrast agent.

11. The method of claim 10, wherein the radiocontrast agent is an iodine-containing radiocontrast agent.

12. The method of claim 10, wherein the radiocontrast agent is diatrizoate, metrizoate, ionic iothalamate, ionic ioxaglate, iopamidol, iohexol, ioxilan, iopromide, iodixanol, ioversol, gadopentetate, gadobenate, gadodiamide, gadoterate, gadoteridol, gadobutrol, or gadoexetate, or a combination thereof.

13. The method of claim 10, wherein the method induces an increase in hepcidin in the human patient.

14. The method of claim 10, wherein the iron composition comprises an iron sucrose composition.

15. The method of claim 14, wherein the iron sucrose composition comprises bicarbonate.

16. The method of claim 14, wherein the iron sucrose composition comprises Fe2+/Fe3+ ratio of approximately 1-10%, a total iron content of 5-19 mg/ml, an organic carbon content of 4-11%, an osmolality of 1100-1600 mOsM/Kg, an iron core size of about 1-3 nm, a Na content of between 0.8%-3%, and an average molecular weight of 10,000-30,000 daltons.

17. The method of claim 14, wherein the iron sucrose composition comprises Fe2+/Fe3+ ratio of approximately 3-4%, a total iron content of 10-15 mg/ml, an organic carbon content of 6-9%, an osmolality of 1400-1580 mOsm/Kg, an iron core size of about 2-2.8 nm, a Na content of between 1%-2%, and an average molecular weight of 20,000-25,000 daltons.

18. The method of claim 14, wherein the iron sucrose composition comprises Fe2+/Fe3+ ratio of approximately about 3.4%, a total iron content of about 12 mg/ml, an organic carbon content of about 7.7%, an osmolality of about 1540 mOsm/Kg, an iron core size of about 2.39 nm, a Na content of about 1.26%, and an average molecular weight of about 23,881 daltons.

19. A method for protecting the kidney during cancer chemotherapy comprising:
administering a chemotherapeutic agent and an amount of protoporphyrin to a human patient, wherein the amount of protoporphyrin confers protection of the patent's kidney against cytotoxic effects of the chemotherapeutic agent.

20-24. (canceled)

25. A method for protecting the kidney during radiocontrast imaging comprising:
administering the radiocontrast agent and an amount of protoporphyrin to a human patient, wherein the amount of protoporphyrin confers protection of the patent's kidney against cytotoxic effects of the radiocontrast agent.

26-30. (canceled)

31. A method for protecting the kidney during cancer chemotherapy comprising:
administering a chemotherapeutic agent and an amount of protoporphyrin and an amount of iron composition to a human patient, wherein the amount of protoporphyrin and the amount of iron composition confers protection of the patent's kidney against cytotoxic effects of the chemotherapeutic agent.

32-41. (canceled)

42. A method for protecting the kidney during radiocontrast imaging comprising:
administering the radiocontrast agent and an amount of protoporphyrin and an amount of iron composition to a human patient, wherein the amount of protoporphyrin and the amount of iron composition confers protection of the patent's kidney against cytotoxic effects of the radiocontrast agent.

43-52. (canceled)
